(19) United States
(12) Plant Patent Application Publication
(10) Pub. No.: US 2019/0313565 P1
(43) Pub. Date: Oct. 10, 2019

(54) Echeveria plant named 'TSAECH1806'

(50) ;

(71) Applicant: Tosjami B.V., Heerhugowaard (NL)

(72) Inventor: Gerard van Langen, Heerhugowaard (NL)

(73) Assignee: Tosjami B.V., Heerhugowaard (NL)

(21) Appl. No.: 16/501,052

(22) Filed: Feb. 13, 2019

(30) Foreign Application Priority Data
Apr. 6, 2018 (QZ) 2018/0956

(51) Int. Cl. A01H 6/00

(52) U.S. Cl. PLT/373

(57) ABSTRACT

A new and distinct Echeveria plant named ‘TSAECH1806’ which is characterized by a dark brown to near-black rosette of succulent foliage which is centered green, as well as the stability of these characteristics from generation to generation.

### CROSS REFERENCE TO RELATED APPLICATIONS

This application claims priority to the Community Plant Variety Rights application number 2018/0956, filed Apr. 6, 2018, which is herein incorporated by reference.

Latin name of the genus and species: The Latin name of the genus and species of the novel variety disclosed herein is Echeveria hybrid.

Variety denomination: The inventive variety of Echeveria disclosed herein has been given the variety denomination ‘TSAECH1806’.

### BACKGROUND OF THE INVENTION

Parentage: ‘TSAECH1806’ is a seedling selection resulting from the controlled pollination of an unnamed and unpatented Echeveria affinis plant, the seed parent, with an unnamed and unpatented Echeveria atropurpurea plant, the pollen parent. The crossing was made by the inventor in the spring of 2015 at a commercial greenhouse in Heerhugowaard, the Netherlands. In the summer of 2016, one seedling resulting from said cross of 2015 was observed to exhibit unique growth and foliage characteristics and was isolated for further evaluation in order to confirm the distinctness and stability of the characteristics first observed. Upon confirmation of distinctness and stability, ‘TSAECH1606’ was selected for commercialization.

Asexual Reproduction: Asexual reproduction of the new cultivar ‘TSAECH1806’, by way of rooting leaf cuttings, was first initiated in the spring of 2017 at the inventor's commercial greenhouse in Heerhugowaard, the Netherlands. Through three subsequent generations, the unique features of this cultivar have proven to be stable and true to type.

### SUMMARY OF THE INVENTION

The cultivar ‘TSAECH1806’ has not been observed under all possible environmental conditions. The phenotype may vary somewhat with variations in environment such as temperature, day length, and light intensity, without, however, any variance in genotype. The following traits have been repeatedly observed and are determined to be the unique characteristics of ‘TSAECH1806’. These characteristics in combination distinguish ‘TSAECH1806’ as a new and distinct Echeveria cultivar:
1. ‘TSAECH1806’ exhibits long, obovate to oblanceolate succulent foliage arranged in a stemless basal rosette; and
2. ‘TSAECH1806’ exhibits a rosette of foliage which is dark brown to near-black foliage, suffused with greyed-purple, and centered yellow-green; and
3. ‘TSAECH1806’ exhibits slightly concave foliage; and
4. ‘TSAECH1806’ exhibits foliage which becomes darker, with less yellow-green coloration at the base, when exposed to high levels ultraviolet light.

### BRIEF DESCRIPTION OF THE FIGURES

FIG. 1 illustrates, as nearly true as it is reasonably possible to make the same in color photographs of this type, an exemplary plant of ‘TSAECH1806’ grown in a commercial greenhouse in Heerhugowaard, the Netherlands. This plant is approximately 12 months old, shown planted in a 10.5 cm container.

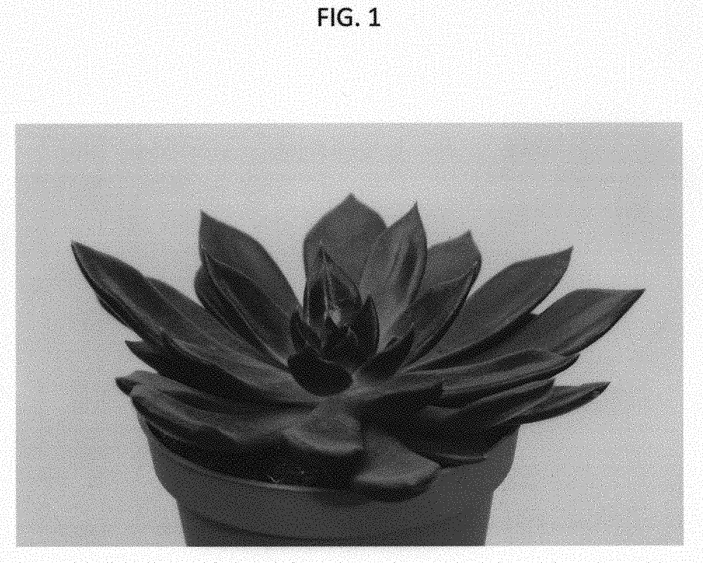

FIG. 2 illustrates, as nearly true as it is reasonably possible to make the same in color photographs of this type, the typical foliage arrangement of ‘TSAECH1806.

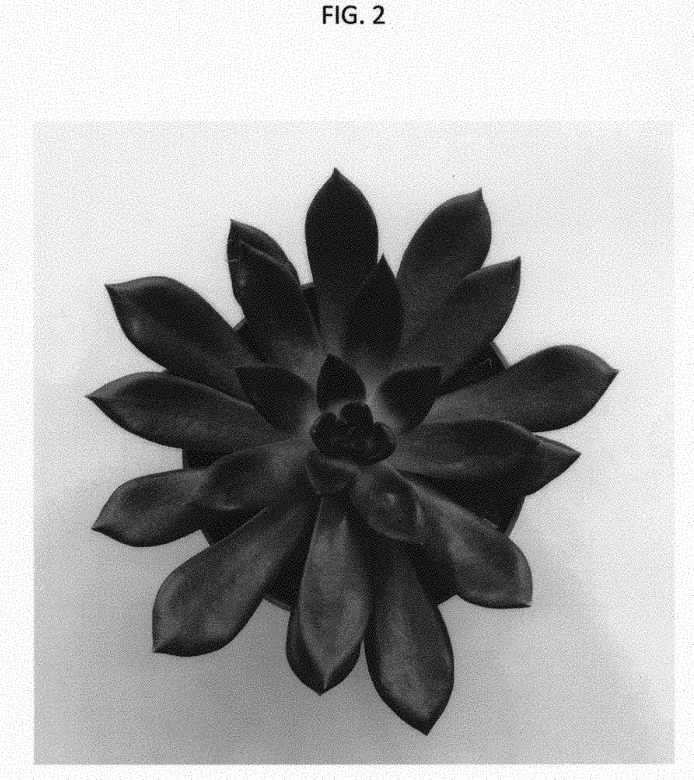

FIG. 3 illustrates, as nearly true as it is reasonably possible to make the same in color photographs of this type, the adaxial surface of the juvenile and mature foliage of ‘TSAECH1806’.

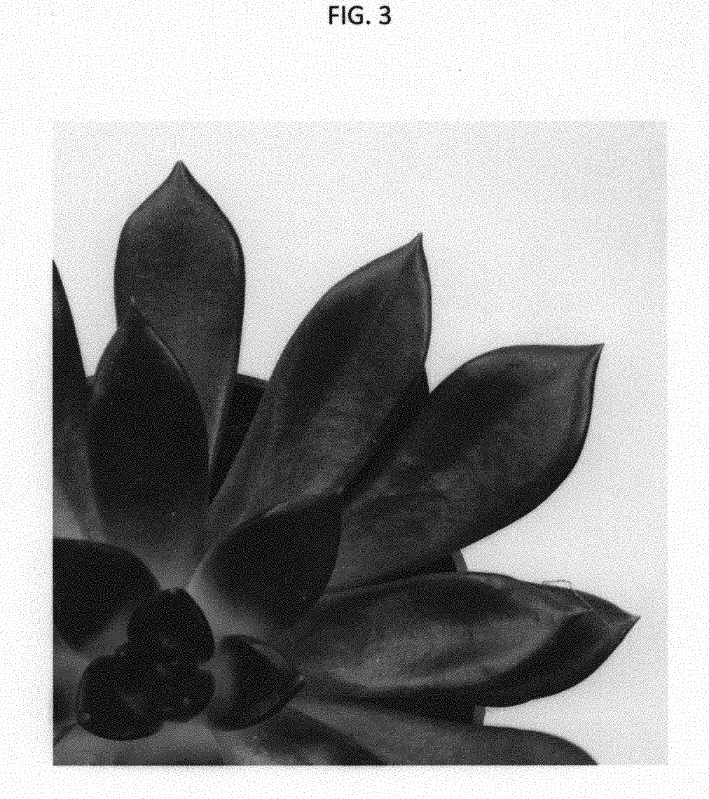

FIG. 4 illustrates, as nearly true as it is reasonably possible to make the same in color photographs of this type, the abaxial surface of the mature foliage of ‘TSAECH1806’.

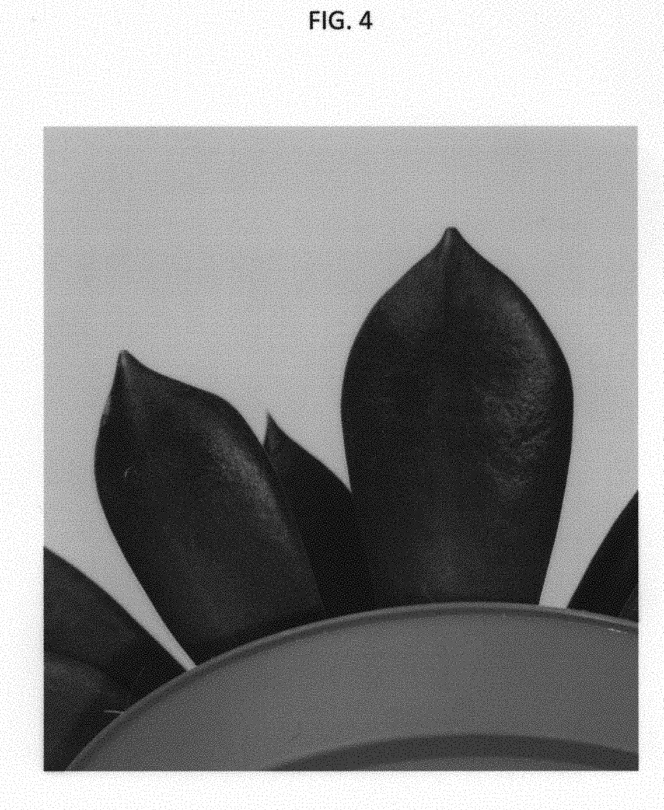

### BOTANICAL DESCRIPTION OF THE PLANT

The following observations and measurements made in December of 2018 describe averages from a sample set of six specimens of 12 months old ‘TSAECH1806’ plants grown in 10.5 cm nursery containers at commercial greenhouse in Heerhugowaard, the Netherlands. Plants were produced using conventional greenhouse production protocols for Echeveria plants which consisted of minimal irrigation and fertilizer applications, and chemical pest and disease control measures against mealy bug and Botrytis as required. Plants were grown under approximately 50 percent shade after propagation and later exposed to full sun once they began to mature. No photoperiodic treatments or artificial light was given to the plants.

Those skilled in the art will appreciate that certain characteristics will vary with older or, conversely, with younger plants. ‘TSAECH1806’ has not been observed under all possible environmental conditions. Where dimensions, sizes, colors and other characteristics are given, it is to be understood that such characteristics are approximations or averages set forth as accurately as practicable. The phenotype of the variety may differ from the descriptions set forth herein with variations in environmental, climatic and cultural conditions. Color notations are based on The Royal Horticultural Society Colour Chart, The Royal Horticultural Society, London, 2015 (sixth edition).

A botanical description of ‘TSAECH1806’ and comparisons with the parent plants and closest known comparator are provided below.
Plant description:
   Growth habit.—Succulent perennial with foliage growing in a stemless basal rosette.
   Plant shape.—Flattened.
   Height from soil level to top of foliar plane.—5.2 cm.
   Plant spread.—Average of 16.2 cm.
   Growth rate.—Moderately fast growing.
   Plant vigor.—Moderately vigorous.
   Propagation.—Type — Leaf cuttings. Time to initiate rooting — Approximately 21 days at 18 degrees Celsius. Crop time — Approximately 30 weeks to produce a marketable plant in a 10.5 cm container.
   Disease and pest resistance or susceptibility.—Neither resistance nor susceptibility to typical Echeveria pests and diseases has been observed.
   Environmental tolerances.—Adapt to, at least, USDA Zones 10 to 12 and temperatures as high as 40 degrees Celsius; moderate tolerance to rain yet drought tolerant once established; high tolerance to wind.
Root system:
   General.—Fine, well-branched fibrous roots.
Foliage:
   Arrangement.—Rosette.
   Division.—Simple.
   Attachment.—Sessile.
   Quantity.—Approximately 30 leaves per rosette.
   Shape.—Obovate to oblanceolate.
   Dimensions.—7.7 cm long, 2.4 cm wide, and 0.6 cm thick, on average.
   Aspect.—Slightly concave and slightly curved upwards.
   Attitude.—Juvenile foliage at the center of the rosette is held outward; foliage becomes progressively more relaxed towards the outer whorls of mature foliage, at an average angle of 15 degrees to horizontal.
   Apex.—Apiculate.
   Base.—Long cuneate.
   Margin.—Entire; lightly undulated.
   Pubescence, texture and luster of the adaxial surface.—Glabrous, smooth, and glossy; glaucous.
   Pubescence, texture and luster of the abaxial surface.—Glabrous, smooth, and gloss; glaucous.
   Color.—Juvenile foliage, adaxial surface — Brown, nearest to RHS 200A, and transitioning to yellow-green towards the base, nearest to a mixture of RHS 145B, 145C, and 145D; the epicuticular wax covering the leaf surface is brown, nearest to RHS 200B. Juvenile foliage, abaxial surface — Brown, nearest to RHS 200B, and transitioning to yellow-green towards the base, nearest to a mixture of RHS 145B, 145C, and 145D; the epicuticular wax covering the leaf surface is brown, nearest to RHS 200B. Mature foliage, adaxial surface — Brown, nearest to in between RHS 200A and 200B, and lightly suffused with greyed-purple, nearest to RHS N186C; transitioning to yellow-green towards the base, nearest to RHS 147D; the epicuticular wax covering the leaf surface is brown, nearest to RHS 200B. Mature foliage, abaxial surface — Greyed-brown, nearest to RHS 187A, and lightly suffused with brown, nearest to RHS 200B; transitioning to yellow-green towards the base, nearest to a mixture of RHS 148C and 148D; the epicuticular wax covering the leaf surface is brown, nearest to RHS 200B.
   Venation.—No venation is visible.
   Petiole.—No petiole; leaves are sessile.

### INFLORESCENCE

No flowering has been observed to date.

### COMPARISONS WITH THE PARENT PLANT AND CLOSEST KNOWN COMPARATOR

Plants of the new cultivar ‘TSAECH1806’ differ from the seed parent, an unnamed Echeveria affinis plant (not patented), in the following characteristics described in Table 1 below.

TABLE 1

| Characteristic | ‘TSAECH1806’ | Seed parent |
|---|---|---|
| Growth habit. | Less compact. | More compact. |
| General coloration | Less yellow-green is | More yellow-green is |
| of the foliage. | present at the leaf | present at the leaf |
| | base; less green color- | base; more green color- |
| | ation at the center of | ation at the center of |
| | the rosette. | the rosette. |

Plants of the new cultivar ‘TSAECH1806’ differ from the pollen parent, an unnamed Echeveria atropurpurea plant (not patented), in the following characteristics described in Table 2 below.

TABLE 2

| Characteristic | ‘TSAECH1806’ | Pollen parent |
|---|---|---|
| Growth habit. | Stemless rosette. | Foliage born on a stem. |
| Foliage aspect. | Slightly concave and | Strongly concave and |
| | slightly curved upwards. | strongly curved upwards. |
| Foliage length. | Longer than the pollen | Shorter than |
| | parent. | ‘TSAECH1806’. |
| General coloration | Generally appearing as | Generally appearing as a |
| of the foliage. | dark brown to black, | mixture of brown and |
| | suffused with greyed- | dark red. |
| | purple. | |
| Foliage, margin | No distinct margin | Very thinly margined |
| color. | coloration. | white. |

### COMPARISONS WITH THE CLOSEST KNOWN COMPARATOR

Plants of the new cultivar ‘TSAECH1806’ differ from the closest known commercial comparator, the common form of Echeveria sp. ‘Black Prince’ (not patented), in the following characteristics described in Table 3 below.

TABLE 3

| Characteristic | ‘TSAECH1806’ | ‘Black Prince’ |
|---|---|---|
| Growth habit. | Stemless rosette. | Foliage born on a stem. |
| Foliage shape. | Slightly concave and | Strongly concave and |
| | slightly curved upwards. | strongly curved upwards. |
| Foliage dimensions. | Longer and more narrow | Shorter and broader than |
| | than ‘Black Prince’. | ‘TSAECH1806’. |
| General coloration | Generally appearing as | Generally appearing as a |
| of the foliage. | dark brown to black, | mixture of dark brown to |
| | suffused with greyed- | black and red. |
| | purple. | |

That which is claimed is:

1. A new and distinct variety of Echeveria plant named ‘TSAECH1806’, substantially as described and illustrated herein.
